(19) United States
(12) Patent Application Publication
(10) Pub. No.: US 2022/0233176 A1
(43) Pub. Date: Jul. 28, 2022

(54) VAGINAL DETECTION METHODS AND KITS USING A TAMPON

(71) Applicant: Anne's Day Ltd., London (GB)

(72) Inventor: VALENTINA MILANOVA, London (GB)

(21) Appl. No.: 17/618,669

(22) PCT Filed: Mar. 13, 2020
(86) PCT No.: PCT/IB2020/052335

§ 371 Date: Dec. 13, 2021

Related U.S. Application Data
(60) Provisional application No. 62/868,270, filed on Jun. 28, 2019.

(51) Int. Cl. A61B 10/00; A61F 13/53; A61F 13/20

(52) U.S. Cl. CPC A61B 10/0045; A61F 13/53; A61F 13/2074; A61F 2013/530036; A61B 2010/0074; A61F 2013/530029; A61F 13/2002

(57) ABSTRACT

The present invention relates to methods of obtaining a biological sample from the vagina and detecting various conditions (e.g., detecting a sexually transmitted infection, determining the composition of the vaginal microbiome, and detecting dysbiosis) using a tampon. The present invention also relates to kits containing a tampon for use in such methods.

### CROSS-REFERENCE TO RELATED APPLICATIONS

The present application claims the benefit under 35 U.S.C. § 119(e) of U.S. Provisional Application No. 62/868,270, filed on Jun. 28, 2019, which is incorporated herein by reference in its entirety for all purposes.

### FIELD OF THE INVENTION

The present invention relates to methods of obtaining a biological sample from the vagina and detecting various conditions (e.g., detecting a sexually transmitted infection, determining the composition of the vaginal microbiome, and/or detecting dysbiosis) using a tampon. The present invention also relates to kits containing a tampon for use in such methods.

### BACKGROUND

Sexually transmitted infections (STIs) remain a major public health challenge in the United States. According to the Centers for Disease Control and Prevention, steep and sustained increases in the number of STIs were observed in the U.S. in 2017. Some STIs have the potential to cause serious health problems for women, such as long-term pelvic or abdominal pain, inability to get pregnant, pregnancy complications, or increased risk of giving or getting Human Immunodeficiency Virus (HIV), especially if not diagnosed and treated early.

The microbial composition of the vagina (i.e., the vaginal microflora, vaginal microbiota or vaginal microbiome) also has a significant role in women's health and the propagation of STIs. Typical microbial colonies in the vagina are benign and beneficial and protect the body from the penetration of pathogenic microbes. Under normal circumstances, beneficial microbial colonies compete with each other for space and resources and maintain a healthy balance of growth. However, when this balance is disturbed (i.e., dysbiosis), microbial colonies have a decreased ability to check each other's growth, leading to overgrowth of certain colonies and damage to smaller beneficial colonies. This results in higher concentrations of waste byproducts from the overgrowth colonies that can overburden the body's waste removal mechanisms. Sustained periods of dysbiosis can negatively impact women's health, such as causing bacterial vaginosis, increase the risk of acquiring STIs, or cause reproductive complications such as spontaneous abortions, premature births, or low fetal birth weight. As such, assessment of a woman's vaginal microbiome is important for preventing and treating such conditions.

Current blood-based methods for diagnosing STIs have reduced patient compliance because they often require a trip to an external facility, where a trained professional can perform venipuncture in a sterile environment. However, venipuncture involves a non-trivial time commitment, travel and labor costs, and often psychological or physical pain that may prevent women from undergoing regular monitoring of blood-based health markers. Other methods for diagnosing STIs or assessing the vaginal microbiome, such as a vaginal swab or brush, do not require a blood sample, but still require a trip to the doctor's office. These methods can result in a lasting painful sensation which may prevent frequent screening and monitoring. Moreover, vaginal swab or brush methods often involve sampling of only a portion of the vagina and can fail to detect bacteria and viruses that are present in different areas of the vaginal canal.

There is a need for improved methods and kits for obtaining a biological sample from the vagina. Such methods and kits could be used, for example, for diagnosing STIs, assessing the vaginal microbiome, or diagnosing dysbiosis, which could lead to improved treatment of the same.

### SUMMARY OF THE INVENTION

In one aspect, the present invention is related to a method for obtaining a biological sample from a subject, comprising obtaining a biological sample from a tampon comprising (i) an absorbent tampon body and (ii) a protective sock around the tampon body; wherein said tampon having been administered to the subject vaginally for a time sufficient to obtain a biological sample on the protective sock.

In another aspect, the present invention is related to a method for obtaining a biological sample from a subject, comprising (a) obtaining a tampon comprising (i) an absorbent tampon body and (ii) a protective sock around the tampon body; wherein said tampon having been administered to the subject vaginally for a time sufficient to obtain a biological sample on the protective sock; and (b) obtaining the biological sample from the protective sock.

In another aspect, the present invention is related to a method to detect a sexually transmitted infection (STI) in a subject, comprising performing a nucleic acid assay on a biological sample to detect and/or quantify the presence of an STI marker; wherein said detecting and/or quantifying of the marker is indicative of an STI in the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

In another aspect, the present invention is related to a method to detect an STI in a subject, comprising (a) obtaining a tampon comprising (i) an absorbent tampon body and (ii) a protective sock around the tampon body; said tampon having been administered to the subject vaginally for a time sufficient to obtain a biological sample on the protective sock; and (b) performing a nucleic acid assay on the biological sample to detect and/or quantify the presence of an STI marker; wherein said detecting and/or quantifying of the marker is indicative of an STI in the subject.

In another aspect, the present invention is related to a method to detect an STI in a subject, comprising (a) administering a tampon to the subject vaginally; wherein the tampon comprises (i) an absorbent tampon body and (ii) a protective sock around the tampon body; (b) maintaining the tampon in the vagina for a time sufficient to obtain a biological sample on the protective sock; and (c) performing a nucleic acid assay on the biological sample to detect and/or quantify the presence of an STI marker; wherein said detecting and/or quantifying of the marker is indicative of an STI in the subject.

In another aspect, the present invention relates to a method to determine the composition of the vaginal microbiome of a subject, comprising performing a nucleic acid assay on a biological sample to determine and/or quantify the vaginal microbiome of the subject; wherein the dominant species of bacteria in the biological sample is indicative of the composition of the vaginal microbiome of the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

In another aspect, the present invention relates to a method to determine the composition of the vaginal microbiome of a subject, comprising (a) obtaining a tampon comprising (i) an absorbent tampon body and (ii) a protective sock around the tampon body; wherein said tampon having been administered to the subject vaginally for a time sufficient to obtain a biological sample on the protective sock; and (b) performing a nucleic acid assay on the biological sample to determine and/or quantify the vaginal microbiome of the subject; wherein the dominant species of bacteria in the biological sample is indicative of the composition of the vaginal microbiome of the subject.

In another aspect, the present invention is related to a method to determine the composition of the vaginal microbiome of a subject, comprising (a) administering a tampon to the subject vaginally; wherein the tampon comprises (i) an absorbent tampon body and (ii) a protective sock around the tampon body; (b) maintaining the tampon in the vagina for a time sufficient to obtain a biological sample on the protective sock; and (c) performing a nucleic acid assay on the biological sample to determine and/or quantify the vaginal microbiome of the subject; wherein the dominant species of bacteria in the biological sample is indicative of the composition of the vaginal microbiome of the subject.

In another aspect, the present invention is related to a method to detect dysbiosis in a subject, comprising performing a nucleic acid assay on a biological sample to detect dysbiosis in the subject; wherein the biological sample is present on a tampon comprising (i) an absorbent tampon body and (ii) a protective sock around the tampon body; wherein said tampon having been administered to the subject vaginally for a time sufficient to obtain a biological sample on the protective sock.

In another aspect, the present invention is related to a method to detect dysbiosis in a subject, comprising (a) obtaining a tampon comprising (i) an absorbent tampon body and (ii) a protective sock around the tampon body; wherein said tampon having been administered to the subject vaginally for a time sufficient to obtain a biological sample on the protective sock; and (b) performing a nucleic acid assay on the biological sample to detect dysbiosis in the subject.

In another aspect, the present invention is related to a method to detect dysbiosis in a subject, comprising (a) administering a tampon to the subject vaginally; wherein the tampon comprises (i) an absorbent tampon body and (ii) a protective sock around the tampon body; (b) maintaining the tampon in the vagina for a time sufficient to obtain a biological sample on the protective sock; and (c) performing a nucleic acid assay on the biological sample to detect dysbiosis in the subject.

In another aspect, the present invention is related to a kit, comprising (a) a tampon comprising (i) an absorbent tampon body and (ii) a protective sock around the tampon body; and (b) instructions for obtaining a biological sample with the tampon.

### BRIEF DESCRIPTION OF THE DRAWINGS

FIG. 1 shows the results of a study comparing a menstrual tampon and a vaginal swab as biological sample collection methods.

FIG. 2 shows additional comparisons from the study of FIG. 1 regarding the type of microorganism identified, or not identified.

FIG. 3 shows the categorization of dysbiosis for each volunteer based on biological samples collected by menstrual tampon or vaginal swab from the study in FIG. 1. Three of the 11 volunteers showed different conclusions with regard to the level of severity of dysbiosis for the different sample collection methods.

FIG. 4 shows a comparison of the amount of biological material collected by three different sample collection methods, i.e., vaginal swab, menstrual tampon, and cervical swab. Samples collected by menstrual tampon more often had the highest amount of biological material compared to the other collection methods tested.

FIG. 5 shows the numbers of identified species (a and b) and total species (c) for three different sample collection methods, i.e., vaginal swab (VS), menstrual tampon (MT), and cervical swab (CS).

FIG. 6 shows a comparison of the diagnostic panels for the Femoflor® 16 and Femoflor® Screen assays.

FIG. 7 shows the diagnostic results of 23 patients using samples collected from three different methods, i.e., vaginal swab, menstrual tampon, and cervical swab, and the Femoflor® Screen assay. The most severe diagnosis from the three collection methods is shown.

### DETAILED DESCRIPTION OF THE INVENTION

In general terms, the present invention is directed to methods of obtaining a biological sample from the vagina and detecting various conditions (e.g., detecting a sexually transmitted infection, determining the composition of the vaginal microbiome, and detecting dysbiosis) using a tampon. In addition, the present invention is directed to kits containing a tampon for use in such methods.

Before explaining embodiments of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### Definitions

Unless defined otherwise, all technical and scientific terms used in this disclosure have the same meanings as commonly understood by one of ordinary skill in the art to which this disclosure belongs.

As used herein, the terms “comprises”, “comprising”, “includes”, “including”, “having”, and their conjugates mean “including but not limited to.” The abbreviation “e.g.” is derived from the Latin exempli gratia, and is used herein to indicate a non-limiting example. Thus, the abbreviation “e.g.” is synonymous with the term “for example.”

It is understood that description herein of an embodiment of the invention as “comprising” certain element(s) includes description of an embodiments as “consisting of” or “consisting essentially of” the same element(s). The term “consisting of” means “including and limited to.” The term “consisting essentially of” means the specified material of a composition, or the specified steps of a method, and those additional materials or steps that do not materially affect the basic characteristics of the material or method.

As used herein, the singular form “a”, “an” and “the” include plural references unless the context clearly dictates otherwise. For example, the term “a compound” or “at least one compound” may include a plurality of compounds, including mixtures thereof.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. Whether or not modified by the term “about”, the claims include equivalents of the recited quantities. In one embodiment, the term “about” means within 10% of the reported numerical value. In another embodiment, the term “about” means within 5% of the reported numerical value.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The term “assay” as used herein refers to the analysis of a sample to determine the presence, absence, quantity or edited nature of one or more components.

The term “tampon” as used herein refers to a feminine hygiene apparatus having an absorbent core and, in some embodiments, a protective sock that covers the absorbent core.

The term “protective sock” as used herein refers to a protective sleeve, which covers the absorbent core of a tampon and prevents fiber loss from inserting and removing the product.

The term “biological sample” as used herein means tissue, cells, blood, fluid and/or mucus contained in the vagina or cervix.

The term “industrial hemp fiber” as used herein means the material isolated from a hemp plant stem. Industrial hemp is a plant known as cannabis sativa L. subsp. sativa var. sativa. The outer stem material of the plant contains bast fibers. Bast fibers can be used to make textiles, for example, and can be blended with other fibers such as flax, cotton, silk or polyester. The inner stem material of the plant contains the hurd, which contains short fibers. These fibers are more woody and typically have industrial applications, such as mulch, animal bedding and litter. Separation of hurd and bast fibers from the stem of the plant is known as decortication. Decortication can occur by water-retting hemp stems and the outer fibers can beaten off the inner core by hand or with a mechanical device. Industrial hemp fiber provides superior absorbance properties for the tampons of the methods and kits of the present invention.

The term “sexually transmitted infection” or “STI” as used herein means infections that are passed from one person to another through sexual contact. STIs can be caused by bacteria, parasites, yeast or viruses. Examples of microbes causing STIs include, but are not limited to, Neisseria gonorrhoeae, Chlamydia trachomatis, Ureaplasma urealyticum, Mycoplasma genitalium, Mycoplasma hominis, syphilis-causing Treponema pallidum, chancroid-causing Haemophilus ducreyi, genital herpes-causing herpes simplex virus 1 and 2, human papillomavirus (HPV), Trichomonas vaginalis and three related organisms Candida albicans, Gardnerella vaginalis and coliform bacteria, and any other examples described herein.

The term “nucleic acid assay” as used herein means a microbiological test to assess a biological sample of the methods described herein. These tests include, but are not limited to, microscopic, bacteriological (e.g., culture based techniques), or molecular based techniques (e.g., polymerase chain reaction (PCR)).

The term “cannabidiol” as used herein refers to 2-[3-methyl-6-(1-methylethenyl)-2-cyclohexen-1-yl]-5-pentyl-1,3-benzenediol, as well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenediol. The synthesis of 2-[3-methyl-6-(1-methylethenyl)-2-cyclohexen-1-yl]-5-pentyl-1,3-benzenediol is described, for example, in Petilka et al., Hely. Chim. Acta, 52:1102 (1969) and Mechoulam et al., J. Am. Chem. Soc., 87:3273 (1965), which are hereby incorporated by reference.

As used herein, “effective amount of cannabidiol” means an amount of cannabidiol that is sufficient to inhibit the occurrence or ameliorate one or more symptoms associated with menstruation.

The terms “microbiota”, “microbiome”, and “microflora” refer to an assemblage of microorganisms localized to an environment. For example, “vaginal microbiota” or “vaginal microflora” are an assemblage of one or more species of microorganisms that are localized to, or found in, a vagina.

The term “dysbiosis” as used herein refers to a microbial imbalance of the vagina, such as an impaired vaginal microbiome.

The term “real-time PCR” as used herein refers to a type of polymerase chain reaction that is based on the amplification of a target DNA sequence. In some embodiments, DNA molecules are heat denatured while the cyclic amplification program proceeds. Target specific primers bind to the denatured DNA templates in the presence of dNTPs and Taq polymerase. Taq polymerase extends the primers, thus providing the synthesis of complementary DNA chains and amplification of target DNA sequence. Real-time PCR technology is based on measurement of fluorescence at every cycle of reaction. The PCR mix contains target-specific hydrolyzing probes bearing reporter and quencher molecules. While the probe is intact, these molecules are close enough to provide effective quenching. Once hybridized to a target sequence, the probe is hydrolyzed by Taq polymerase. Thereby reporter and quencher become separated and fluorescence increases proportionally to target sequence amplification. The intensity of fluorescence is analyzed with a real-time PCR instrument data collection unit and the software provided.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Throughout this application, various embodiments of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5 and 6. This applies regardless of the breadth of the range.

### Tampons and Biological Samples

The methods and kits of the present invention relate to the use of a tampon for obtaining a biological sample and/or for detecting certain conditions (e.g., detecting a sexually transmitted infection (STI), determining the composition of the vaginal microbiome, and/or detecting dysbiosis). The following aspects of the tampons and biological samples of the present invention are applicable to any of the methods and kits of the present invention described herein.

In some embodiments, a tampon for the methods and kits of the present invention comprises (i) an absorbent tampon body and (ii) a protective sock around the tampon body. In some embodiments, the absorbent tampon body is made of industrial hemp fiber. In some embodiments, the absorbent tampon body is made of industrial hemp fiber strips. In other embodiments, the protective sock of a tampon is made of industrial hemp fiber. In some embodiments, the absorbent tampon body and/or protective sock is made of industrial hemp fiber and cotton.

In other embodiments, the tampon body and/or the protective sock of the tampon is made of traditional tampon materials, such as rayon, viscose, and/or cotton.

In some embodiments, the tampon comprises a pharmaceutical composition comprising an effective amount of cannabidiol (CBD). In some embodiments, the pharmaceutical composition is applied to the surface of the tampon body. In some embodiments, the tampon contains a strip comprising the pharmaceutical composition. In some embodiments, the strip is positioned on the tampon body. In some embodiments, the tampon contains a cylindrical cup (e.g., about ¼ inch in diameter) that encapsulates the pharmaceutical composition. In some embodiments, the cup surrounds a circumference of the proximal end of the tampon body. In some embodiments, the pharmaceutical composition comprises cocoa oil. In some embodiments, the effective amount of the CBD is from about 2 mg to about 200 mg, or any value or range or values therein.

In other embodiments, the tampon contains an applicator.

In other embodiments, the tampon is a menstrual tampon.

Additional embodiments of a tampon that can be used in the methods and kits of the present invention are described, for example, in U.S. Provisional Appl. No. 62/714,656, which is incorporated by reference herein in its entirety. Methods of making such tampons are also known and described, for example, in U.S. Provisional Appl. No. 62/714,656, which is incorporated by reference herein in its entirety.

The tampons of the present disclosure have several advantages for use in the methods and kits of the present invention. First, they provide a convenient and reliable sampling device for obtaining a biological sample or for the methods and kits described herein (e.g., detecting an STI, determining the composition of the vaginal microbiome, and/or detecting dysbiosis). Unlike a vaginal swab or brush, a tampon can travel throughout the entirety of the vaginal canal, collecting a sample from its various, difficult to reach regions.

Second, the tampons of the present disclosure can function both for collecting a biological sample and at the same time for delivering CBD. Since CBD is released in the vaginal canal, where a pH of 3.8-4.5 is normal, CBD is not destroyed in such an environment. The CBD will absorb across the mucous lining on the vaginal wall and enter the bloodstream directly. As a result, the amount of CBD remains higher compared to oral administration where the amount of active agent is reduced, for example, due to first pass liver metabolism.

Third, organic industrial hemp is grown without the need for pesticides, herbicides, and fertilizers, making the crop truly organic and thus safer to use inside the female body. Hemp requires no irrigation, apart from rainwater, meaning the crop has no environmental footprint. Industrial hemp is also biodegradable, which means users can flush the used product, without worrying about pollution.

Fourth, tampons containing CBD can be manufactured in a certified clean room facility to ensure no harmful bacteria are found on the surface of the product. Gamma rays can also be used to sterilize the product in its packaging. This significantly reduces the risk of foreign bacteria being introduced into the vaginal canal and thus lowers the instances of Toxic Shock Syndrome (TSS).

The tampons of the methods and kits of the present invention are used to obtain a biological sample from a subject. To this end, a tampon is inserted into the vagina of the subject to obtain a biological sample. In some embodiments, the tampon is administered and/or removed by the subject. In other embodiments, the tampon is administered and/or removed from the subject by a health care provider.

In some embodiments, the tampon is left in the vagina for a time sufficient to collect the biological sample. In some embodiments, the time sufficient to collect the sample is about 1, 5, 15, 10, 20, 25, 30, 35, 40, 45, 50, 55 or 60 minutes, or any range of values therein. In some embodiments, the subject is menstruating. In other embodiments, the subject is not menstruating.

In some embodiments, the biological sample is tissue, cells, blood, fluid and/or mucus contained in the vagina or cervix. In some embodiments, the biological sample is obtained on the protective sock of the tampon. In some embodiments, the biological sample is obtained on the absorbent body of the tampon. In some embodiments, the biological sample is obtained on the protective sock of the tampon and the absorbent body of the tampon.

In some embodiments, a biological sample collected using a tampon of the present invention is evaluated by a nucleic acid assay. In some embodiments, the biological sample is evaluated immediately following removal of the tampon from the vagina of the subject. In other embodiments, the biological sample is evaluated at least 1, 2, 3, 4, 5, 6, 7, 8, 9, 10, 11, 12, 13, 14, 15, 20, 25 or 30 days after the tampon is removed from the vagina. In other embodiments, the biological sample is collected from the tampon (e.g., from the protective sock and/or the absorbent body, e.g., by removing the sock or body, or a sample thereof, from the rest of the tampon) before it is evaluated by nucleic acid assay.

In some embodiments, the biological sample is stored at room temperature at least 1, 2, 3, 4, 5, 6, 7, 8, 9, 10, 11, 12, 13, 14, 15, 20, 25 or 30 days after the tampon is removed from the vagina. In other embodiments, the biological sample is stored at a temperature lower than room temperature, such about −80, −20, −4, 0, 4, or 12 degrees Celsius after the tampon is removed from the vagina.

The methods of kits of the present invention have the advantage of allowing biological samples to be stored at room temperature for significant periods of time before they are evaluated by nucleic acid assay. This allows for a more convenient experience for a subject in need of collecting a vaginal biological sample, because, for example, the subject can collect the biological sample themselves at home and then mail the sample to a laboratory for further evaluation. In addition, the methods of kits of the present invention allow a biological sample to be collected and processed later without concern of bacteria growing on the sample during transit of the sample from the subject to the laboratory.

### Methods for Obtaining a Biological Sample

In some embodiments, the present invention is directed to a method for obtaining a biological sample from a subject. In some embodiments, the method comprises obtaining a biological sample from a tampon comprising (i) an absorbent tampon body and (ii) a protective sock around the tampon body. In some embodiments, the method results in collection of a greater amount of biological material compared to the amount of biological material collected by a conventional collection method (e.g., vaginal swab or cervical swab). In some embodiments, the tampon comprises CBD.

In other embodiments, the present invention is directed to a method for obtaining a biological sample from a subject, comprising (a) obtaining a tampon comprising (i) an absorbent tampon body and (ii) a protective sock around the tampon body; wherein said tampon having been administered to the subject vaginally for a time sufficient to obtain a biological sample on the protective sock; and (b) obtaining the biological sample from the protective sock. In some embodiments, the method further comprises separating the protective sock from the tampon before obtaining the biological sample from the protective sock.

In other embodiments, the present invention is directed to a method for obtaining a biological sample from a subject, comprising (a) administering a tampon to the subject vaginally; wherein the tampon comprises (i) an absorbent tampon body and (ii) a protective sock around the tampon body; and (b) maintaining the tampon in the vagina for a time sufficient to obtain a biological sample on the protective sock. In some embodiments, the method further comprises (c) removing the tampon from the vagina. In some embodiments, the method further comprises (d) separating the protective sock from the tampon. In some embodiments, the method further comprises (e) obtaining the biological sample from the protective sock.

In some embodiments, the tampon has been administered to the subject vaginally for a time sufficient to obtain a biological sample on the absorbent body and/or protective sock. In some embodiments, the time sufficient to collect the sample is about 1, 5, 15, 10, 20, 25, 30, 35, 40, 45, 50, 55 or 60 minutes, or any range of values therein. In some embodiments, the subject is menstruating. In other embodiments, the subject is not menstruating.

As described further herein, the methods of kits of the present invention have the advantage of convenience in biological sample collection, storage, transport, processing, and analysis, and superior detection results, as compared to the conventional methods, such as vaginal swabs or cervical swabs.

### Methods for Detecting an STI

In some embodiments, the present invention is directed to a method to detect an infection (e.g., a sexually transmitted infection (STI)) in a subject. In some embodiments, the method comprises performing a nucleic acid assay on a biological sample to detect and/or quantify the presence of an STI marker; wherein said detecting and/or quantifying of the marker is indicative of an STI in the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

In some embodiments, the present invention is directed to a method to detect an STI in a subject, comprising (a) obtaining a tampon comprising (i) an absorbent tampon body and (ii) a protective sock around the tampon body; wherein said tampon having been administered to the subject vaginally for a time sufficient to obtain a biological sample on the protective sock; and (b) performing a nucleic acid assay on the biological sample to detect and/or quantify the presence of an STI marker; and wherein said detecting of the marker is indicative of an STI in the subject.

In some embodiments, the present invention is directed to a method to detect an STI in a subject, comprising (a) administering a tampon to the subject vaginally; wherein the tampon comprises (i) an absorbent tampon body and (ii) a protective sock around the tampon body; (b) maintaining the tampon in the vagina for a time sufficient to obtain a biological sample on the protective sock; and (c) performing a nucleic acid assay on the biological sample to detect and/or quantify the presence of an STI marker; wherein said detecting and/or quantifying of the marker is indicative of an STI in the subject.

In some embodiments, the tampon has been administered to the subject vaginally for a time sufficient to obtain a biological sample on the absorbent body and/or protective sock. In some embodiments, the time sufficient to collect the sample is about 1, 5, 15, 10, 20, 25, 30, 35, 40, 45, 50, 55 or 60 minutes, or any range of values therein. In some embodiments, the subject is menstruating. In other embodiments, the subject is not menstruating.

### Nucleic Acid Assays

In some embodiments, the method further comprises separating the absorbent body and/or protective sock from the tampon before performing the nucleic acid assay. In some embodiments, the method further comprises isolating the biological sample from the absorbent body and/or protective sock before performing the nucleic acid assay.

In some embodiments, a nucleic acid assay is performed on the biological sample.

In some embodiments, the nucleic acid assay is polymerase chain reaction (PCR). In such embodiments, the PCR can be real-time PCR or quantitative PCR.

In such embodiments, the PCR can comprise isolation of DNA from the biological sample, amplification of a DNA sequence of an STI marker, and detection and/or quantification of the amplified sequence. Methods for isolation of DNA from a biological sample are known and described, for example, in the Examples, Maaroufi et al., J Clin Microbiol. 2004; 42(7): 3159-3163; and Butler et al. Adv Topics Forensic DNA Typing: Methodology, 2012. Such methods generally involve lysis of the sample (e.g., with a salt solution, detergent or Proteinase K), followed by DNA purification (e.g., separation with organic solvents, or alkaline or heat denaturation). Kits and protocols for isolating DNA are also available for isolation of DNA, such as, for example, the PREP-NA PLUS DNA/RNA Extraction Kit (P-002/2EU), PREP-GS PLUS DNA Extraction Kit (P-003/2EU), and PREP-RAPID DNA Extraction Kit (P-001/1EU) (DNA-Technology & Production, Russia).

Methods for performing PCR (e.g., DNA amplification, detection and quantification) are also known and described, for example, in the Examples, Garibyan et al., J Invest Dermatol. 2013; 133(3):e6; and Walker-Daniels, Mater Methods 2012; 2:119. Such methods generally involve amplification of a target nucleic acid sequence via the use of a DNA polymerase, primers, and nucleotides. The template for a PCR reaction may be any nucleic acid sequence of interest (e.g., an STI marker or microorganism associated with an STI) and the nucleic acid source may be DNA, RNA, or cDNA. Primers are short sequences of nucleotides typically synthesized in vitro. They are designed to anneal to opposite strands of a specific nucleic acid template target and usually are between 15-40 bases long. A variety of DNA polymerases have been utilized for PCR, such as Taq DNA polymerase. This enzyme adds the deoxyribonucleoside triphosphates (dNTPs or nucleotides) onto the ends of the primers to extend the nucleic acid based on the template nucleic acid sequence.

A PCR reaction mixture is typically temperature cycled, typically 20-40 times. Denaturation of a nucleic acid template sequence is achieved at 95° C. Then, the temperature is cooled to 37-60° C. to anneal primers to the target sequence. Extension of the primers with nucleotides by the DNA polymerase is achieved at temperatures ranging from 60-72° C. Conventional cycling conditions are 95° C. for 5 minutes initially to denature all template nucleic acid, followed by 2-40 repeats of 95° C. for 30 sec, 60° C. for 30 sec and 72° C. for 1 minute. The time spent at each temperature can be optimized for specific assays.

Typically, a PCR reaction occurs in three phases. The exponential phase is the period in which exact doubling of a nucleic acid product occurs every cycle. Real-time PCR detection is carried out during this exponential phase. The linear phase occurs as the reaction is slowing due to the consumption of the reagents and the degradation of the products. The final stage is the plateau phase, which occurs when the reaction has stopped, and no additional amplicon is being generated. This is the point at which the PCR reaction product can be detected and analyzed.

Detection of a PCR product can be done in many ways. A common method of detection is visualization via agarose gel electrophoresis. This involves separating nucleic acid fragments via electrophoresis and staining the nucleic acid with an intercalating dye such as ethidium bromide or SybrSafe and detection using a UV light source and imaging system. Real-time PCR uses specialized thermocyclers that detect the fluorescent signal in each well. This signal is indicative of the amount of double-stranded nucleic acid within the reaction tube or well. This signal, in relative fluorescent units, can be plotted by the thermocycler software versus cycle number and quantitated.

This description related to nucleic acid and PCR assays for the methods of the present invention is applicable to the other methods described herein.

### Methods of Determining Markers or Microorganisms Associated with STIs

In some embodiments, methods of the present invention are directed to using a nucleic acid assay (e.g., PCR) to detect one or more markers for an STI or one or more microorganisms associated with an STI. In some embodiments, the STI marker is one or more aerobic microorganisms. In some embodiments, the STI marker is one or more anaerobic microorganisms. In some embodiments, the STI marker is a bacteria, virus, yeast, mycoplasma, or ureaplasma. In some embodiments, the STI marker is a bacteria of the phyla Firmicutes, Actinobacteria, Bacteroidetes, Fusobacteria, or Proteobacteria.

In some embodiments, the STI is bacterial vaginosis (BV), chlamydia, herpes, genital herpes, HSV-1, HSV-2, hepatitis B, trichomoniasis, human immunodeficiency virus (HIV), acquired immunodeficiency syndrome (AIDS), human papilloma virus (HPV), chancroid, molluscum contagiosum, scabies, syphilis, gonorrhea, or crabs. Examples of microbes causing STDs include, but are not limited to, Neisseria gonorrhoeae, Chlamydia trachomatis, Ureaplasma urealyticum, Mycoplasma genitalium, Mycoplasma hominis, syphilis-causing Treponema pallidum, chancroid-causing Haemophilus ducreyi, Trichomonas vaginalis, and three related organisms Candida albicans, Gardnerella vaginalis and coliform bacteria.

In other embodiments, the STI is bacterial vaginosis (BV). BV is a condition that happens when there is too much of certain bacteria in the vagina. This changes the normal balance of bacteria in the vagina. BV is the most common vaginal infection in women ages 15-44. The cause of this condition is not known but the infection typically occurs in sexually active women.

In some embodiments, the STI is chlamydia. Chlamydia, caused by infection with Chlamydia trachomatis, is a common sexually STD that can cause serious, permanent damage to a woman's reproductive system and cause difficult or impossible for future pregnancy. It can cause cervicitis in women and can lead to serious consequences including pelvic inflammatory disease (PID), tubal factor infertility, ectopic pregnancy, and chronic pelvic pain. Chlamydia can also cause a potentially fatal ectopic pregnancy (pregnancy that occurs outside the womb).

In some embodiments, the STI is genital herpes. Genital herpes is an STI caused by the herpes simplex virus type 1 (HSV-1) or type 2 (HSV-2). Genital herpes can cause painful genital ulcers that can be severe and persistent in persons with suppressed immune systems, such as HIV-infected persons. Both HSV-1 and HSV-2 can also cause rare but serious complications such as aseptic meningitis (inflammation of the linings of the brain). Development of extragenital lesions (e.g., buttocks, groin, thigh, finger, or eye) can occur during the course of infection. There are also potential complications for a pregnant woman and her newborn child.

In some embodiments, the STI is gonorrhea. Gonorrhea is an STI caused by infection with the Neisseria gonorrhoeae bacterium. N. gonorrhoeae infects the mucous membranes of the reproductive tract, including the cervix, uterus, and fallopian tubes in women, and the urethra in women and men. In women, gonorrhea can spread into the uterus or fallopian tubes and cause pelvic inflammatory disease (PID).

In some embodiments, the STI is human papillomavirus (HPV) or pelvic inflammatory disease (PID).

In some embodiments, the STI is syphilis. Syphilis is an STI caused by the bacterium Treponema pallidum. Transmission of syphilis can occur during vaginal, anal, or oral sex. In addition, pregnant women with syphilis can transmit the infection to their unborn child.

In some embodiments, the STI is trichomoniasis. Trichomoniasis (or “trich”) is a very common STI. It is caused by infection with a protozoan parasite called Trichomonas vaginalis. Trichomoniasis can increase the risk of getting or spreading other sexually transmitted infections. For example, trichomoniasis can cause genital inflammation that makes it easier to get infected with HIV, or to pass the HIV virus on to a sex partner.

Methods and materials for assessing such STI markers and microorganisms associated with an STI by a nucleic acid assay (e.g., PCR) are known and described, for example, in the Examples, and U.S. Pat. Nos. 10,283,217 and 9,916,428.

The methods of the present invention have the advantage of ease of use (e.g., lab workers can conduct STI tests by removing the protective sock from a tampon rather than having to cut the tampon in pieces; and/or STI tests can be done in a commercial laboratory environment, rather than in a research laboratory environment). As shown in the Examples, the methods of the present invention also have the advantage of improved accuracy for screening, for example, an STI. These advantages are applicable to all of the methods and kits described herein.

### Methods for Determining the Composition of the Vaginal Microbiome

In some embodiments, the present invention is directed to a method to determine the composition of the vaginal microbiome of a subject. In some embodiments, the method comprises performing a nucleic acid assay on a biological sample to determine and/or quantify the vaginal microbiome of the subject; wherein the dominant species of bacteria in the biological sample is indicative of the composition of the vaginal microbiome of the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

In some embodiments, the present invention is directed to a method to determine the composition of the vaginal microbiome of a subject, comprising (a) obtaining a tampon comprising (i) an absorbent tampon body and (ii) a protective sock around the tampon body; wherein said tampon having been administered to the subject vaginally for a time sufficient to obtain a biological sample on the protective sock; and (b) performing a nucleic acid assay on the biological sample to determine and/or quantify the vaginal microbiome of the subject; wherein the dominant species of bacteria in the biological sample is indicative of the composition of the vaginal microbiome of the subject.

In some embodiments, the present invention is directed to a method to determine the composition of the vaginal microbiome of a subject, comprising (a) administering a tampon to the subject vaginally; wherein the tampon comprises (i) an absorbent tampon body and (ii) a protective sock around the tampon body; (b) maintaining the tampon in the vagina for a time sufficient to obtain a biological sample on the protective sock; and (c) performing a nucleic acid assay on the biological sample to determine and/or quantify the vaginal microbiome of the subject; wherein the dominant species of bacteria in the biological sample is indicative of the composition of the vaginal microbiome of the subject.

In some embodiments, the method further comprises separating the protective sock from the tampon before performing the nucleic acid assay. In some embodiments, the method further comprises isolating the biological sample from the protective sock before performing the nucleic acid assay.

In some embodiments, the tampon has been administered to the subject vaginally for a time sufficient to obtain a biological sample on the absorbent body and/or protective sock. In some embodiments, the time sufficient to collect the sample is about 1, 5, 15, 10, 20, 25, 30, 35, 40, 45, 50, 55 or 60 minutes, or any range of values therein. In some embodiments, the subject is menstruating. In other embodiments, the subject is not menstruating.

In some embodiments, the nucleic acid assay of such methods is polymerase chain reaction (PCR). In such embodiments, the PCR can be real-time PCR or quantitative PCR. In such embodiments, the PCR can comprise isolation of DNA from the biological sample, amplification of total bacterial DNA from the biological sample, and detection and/or quantification of the dominant species of bacteria in the biological sample. Methods for isolation of DNA and performing PCR are known and described further herein. In addition, kits and protocols for determining the composition of the vaginal microbiome are also known and described, for example, in the Examples (e.g., Femoflor® Screen Real-Time PCR Detection Kit)

In some embodiments, amplification of total bacterial DNA can utilize broadly reactive primers described in the Examples and in, for example, Table 2 using at least one forward primer and at least one reverse primer. In one embodiment, up to five forward and up to five reverse primers can be used simultaneously to amplify total bacterial DNA from samples. In certain embodiments, one, two, three, four, or five forward primers and one, two, three, four, or five reverse primers are used simultaneously. In one embodiment, five forward and five reverse primers are used simultaneously to amplify total bacterial DNA from a sample. More than five forward and/or reverse primers can also be used. In one embodiment, up to 100 forward primers and up to 100 reverse primers can be used, or any integer in between five and 100 including, for example, 10, 15, 20, 30, 40, 50, 60, 70, 80 or 90.

In some embodiments, the methods of the present invention assess the relative abundance or ratio of dominant versus non-dominant bacterial species within the biological sample. In some embodiments, the methods of the present invention can also assess the relative abundance or ratio of a non-dominant bacterial species compared to another non-dominant bacterial species within the biological sample. In some embodiments, the methods of the present invention also allow the identification and/or characterization of non-dominant species using melt curve analysis. The identification, characterization, relative abundance, or ratio of non-dominant species can be used to inform treatment decisions including whether to treat, the type of treatment, changing treatment and/or the length of treatment.

In some embodiments, the nucleic acid assay used in such methods can employ traditional PCR-based strategies. These strategies include, for example, the 16S clone and sequence approach (16S-C&S; culture and sensitivity) that uses broad spectrum primers to generate a complex amplicon of the 16S rDNA gene of bacterial species, which is then cloned into E. coli and 100 to 1000 clones are sequenced. The bacterial species are typically determined by comparison to large 16S rDNA databases.

In some embodiments, the nucleic acid assay of the present methods can also use custom primers and/or blocking primers to selectively amplify bacterial DNA within a vaginal microbiome. This approach has been described, for example, in U.S. Pat. No. 10,253,377.

### Methods to Detect or Diagnose Dysbiosis

In some embodiments, the present invention is directed to a method to detect or diagnose dysbiosis in a subject. In some embodiments, the method comprises performing a nucleic acid assay on a biological sample to detect dysbiosis in the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

In some embodiments, the present invention is directed to a method to detect dysbiosis in a subject, comprising (a) obtaining a tampon comprising (i) an absorbent tampon body and (ii) a protective sock around the tampon body; wherein said tampon having been administered to the subject vaginally for a time sufficient to obtain a biological sample on the protective sock; and (b) performing a nucleic acid assay on the biological sample to detect dysbiosis in the subject.

In some embodiments, the present invention is directed to a method to detect dysbiosis in a subject, comprising (a) administering a tampon to the subject vaginally; wherein the tampon comprises (i) an absorbent tampon body and (ii) a protective sock around the tampon body; (b) maintaining the tampon in the vagina for a time sufficient to obtain a biological sample on the protective sock; and (c) performing a nucleic acid assay on the biological sample to detect dysbiosis in the subject.

In some embodiments, the tampon has been administered to the subject vaginally for a time sufficient to obtain a biological sample on the absorbent body and/or protective sock. In some embodiments, the time sufficient to collect the sample is about 1, 5, 15, 10, 20, 25, 30, 35, 40, 45, 50, 55 or 60 minutes, or any range of values therein. In some embodiments, the subject is menstruating. In other embodiments, the subject is not menstruating.

In some embodiments, the method further comprises separating the protective sock from the tampon before performing the nucleic acid assay. In some embodiments, the method further comprises isolating the biological sample from the protective sock before performing the nucleic acid assay. Methods for isolating DNA and performing a nucleic acid assay (e.g., PCR) are known and described further herein.

Vaginal dysbiosis is a condition characterized by the imbalance of the qualitative and quantitative composition of the microbiota. Dysbiotic disorders are differentiated according to their severity into moderate dysbiosis, where the proportion of Lactobacilli is within the range of 20-80% of total bacterial load (TBL), and apparent dysbiosis, where the proportion of lactobacilli is below 20% of the TBL. Depending on the prevailing group of the opportunistic microorganisms, aerobic, anaerobic, and mixed aerobic-anaerobic dysbiosis can also be distinguished.

In some embodiments, the nucleic acid assay of such methods is polymerase chain reaction (PCR). In such embodiments, the PCR can be real-time PCR or quantitative PCR. In such embodiments, the PCR can comprise isolation of DNA from the biological sample, amplification of a DNA sequence of a marker for dysbiosis, and detection and/or quantification of the amplified sequence.

In some embodiments, the marker for dysbiosis being detected and/or quantified can be one or more of a total bacterial mass (TBM), Lactobacillus spp., Enterobacterium spp., Streptococcus spp., Staphylococcus spp., Gardnerella vaginalis/Prevotella bivia/Porphyromonas spp., Eubacterium spp., Sneathia spp./Leptotrihia spp./Fusobacterium spp., Megasphaera spp./Veilonella spp./Dialister spp., Lachnobacterium spp./Clostridium spp., Mobiluncus spp./Corynebacterium spp., Peptostreptococcus spp., Atopobium vaginae, Mycoplasma hominis, Ureaplasma (urealyticum+parvum), Mycoplasma genitalium, or Candida spp. In some embodiments, the marker for dysbiosis being detected and/or quantified can be one or more of a total bacterial mass (TBM), Lactobacillus spp., Gardnerella vaginalis/Prevotella bivia/Porphyromonas spp., Candida spp., Mycoplasma hominis, Ureaplasma (urealyticum+parvum), Mycoplasma genitalium, Trichomonas vaginalis, Neisseria gonorrhoeae, Chlamydia trachomatis, HSV-2, cytomegalovirus (CMV), and HSV-1.

In some embodiments, dysbiosis is indicated in the subject when less than about 80% of bacteria in the biological sample is Lactobacillus. In such embodiments, the Lactobacillus can be L. crispatus, L. iners, L. gasseri, or L. jensenii. In some embodiments, the dysbiosis can be moderate dysbiosis. In such embodiments, the moderate dysbiosis can be indicted in the subject when from about 20% to about 80% of bacteria in the biological sample is Lactobacillus.

In some embodiments, the dysbiosis is apparent dysbiosis, apparent mixed dysbiosis, moderate mixed dysbiosis, apparent anaerobic dysbiosis, or moderate anaerobic dysbiosis.

In some embodiments, the dysbiosis is apparent dysbiosis. In such embodiments, apparent dysbiosis is indicated in the subject when less than about 20% of bacteria in the biological sample of the subject is Lactobacillus.

In some embodiments, the dysbiosis is apparent anaerobic dysbiosis. In some embodiments, apparent anaerobic dysbiosis is correlated with the presence of BV in the subject. In some embodiments, apparent anaerobic dysbiosis is associated A. vaginae in the subject and/or the subject has recurrent BV.

In some embodiments, the dysbiosis is aerobic dysbiosis. Aerobic dysbiosis is clinically manifested as aerobic vaginitis (AV), characterized by the classic symptoms of inflammation of the vaginal mucosa, exocervix, and vulva (hyperemia, erosion of mucous membranes, pathological leucorrhoea, discomfort, itching, and discharge from the genital tract).

In some embodiments, the dysbiosis is apparent aerobic dysbiosis associated primarily with streptococci. In other embodiments, the dysbiosis is apparent aerobic dysbiosis, primarily associated with enterobacteria.

In some embodiments, the dysbiosis is apparent mixed dysbiosis. Clinically apparent mixed (aerobic-anaerobic) dysbiosis can manifest as both BV and AV.

### Kits

In some embodiments, the present invention is directed to a kit useful for a method of the present invention described herein.

In some embodiments, the kit comprises (a) a tampon comprising (i) an absorbent tampon body and (ii) a protective sock around the tampon body; and (b) instructions for obtaining a biological sample with the tampon. In some embodiments, the tampon is any embodiment of tampon described herein. For example, in some embodiments, the tampon body of the tampon is made of industrial hemp fiber. In some embodiments, the tampon body is made of industrial hemp fiber and cotton. In other embodiments, the tampon further comprises a pharmaceutical composition comprising an effective amount of CBD.

In some embodiments, the kit further comprises a container for storing the tampon after a biological sample is collected. In some embodiments, the container is a plastic or glass vial.

In other embodiments, the kit further comprises a self-assessment survey. In some embodiments, the self-assessment survey asks the subject question(s) about one of more of the following: sexual behavior, eating habits, smoking, stress, alcoholic drinking, exercise, contraceptive practices, depression, and anxiety.

In other embodiments, the kit further comprises a mailing envelope for shipping the tampon to a laboratory or doctor's office after a biological sample is collected. In some embodiments, the mailing envelope has pre-paid postage.

Embodiments of the present disclosure can be further defined by reference to the following non-limiting examples. It will be apparent to those skilled in the art that many modifications, both to materials and methods, may be practiced without departing from the scope of the present disclosure.

### EXAMPLES

The following methods and materials were used for the studies described in the examples. The methods were developed from the manufacturer's instructions for the Femoflor® 16 Real-Time PCR Detection Kit or Femoflor® Screen Real-Time PCR Detection Kit (DNA-Technology Research & Production, Russia).

### Processing and Storage of the Tampon Samples

The processing and storage of tampons containing vaginal biological samples was performed in accordance with the manufacturer's instructions for the PREP-NA PLUS or PREP-GS PLUS DNA extraction kits (DNA-Technology Research & Production, Russia). Tampons containing vaginal biological samples were collected, as described further in the examples below. The protective socks, immersed with the vaginal biological samples, were separated from the tampons. The cottons laced with the scrapped vaginal biological samples were removed from the swabs. The samples were then transferred to labelled individual plastic tubes containing 300 μl of sterile physiological saline solution or to labelled individual plastic tubes containing PREP-RAPID DNA Extraction Kit (P-001/1EU) solution (DNA-Technology Research & Production, Russia). The individually contained sample materials were stored at temperatures between 2° C. and 8° C. until analysis. All components of the Femoflor® 16 Real-Time PCR Detection Kit or Femoflor® Screen Real-Time PCR Detection Kit were stored at a temperature of 2° C. to 8° C. over the storage period. Paraffin-sealed PCR-mixes were stored in a dark place over the storage period.

### Isolation of DNA

Following storage, total DNA was isolated from the samples using the PREP-NA PLUS DNA/RNA Extraction Kit (P-002/2EU), PREP-GS PLUS DNA Extraction Kit (P-003/2EU), or PREP-RAPID DNA Extraction Kit (P-001/1EU) (DNA-Technology & Production, Russia). If the sample was stored in sterile physiological saline solution, the PREP-NA PLUS DNA/RNA Extraction Kit (P-002/2EU) or PREP-GS PLUS DNA Extraction Kit (P-003/2EU) were used. If the sample was stored in PREP-RAPID DNA Extraction Kit (P-001/1EU) reagent, the PREP-NA PLUS DNA/RNA Extraction Kit was used.

For each analysis, a negative control (blank sample that passed all stages of DNA isolation procedure) was prepared. Transport medium or sterile physiological saline was used as a negative control in the volume according to the manufacturer's instructions for the DNA extraction kit.

### Real-Time PCR

Marked strips with paraffin sealed PCR-mix from the Femoflor® 16 Real-Time PCR Detection Kit or Femoflor® 16 Real-Time PCR Detection Kit were used to assay test samples, positive control (C+) and negative control (C−). The Femoflor® 16 Real-Time PCR Detection Kit was designed to target one or more of the following characteristics or strains: total bacterial mass (TBM), Lactobacillus spp., Enterobacterium spp., Streptococcus spp., Staphylococcus spp., Gardnerella vaginalis/Prevotella bivia/Porphyromonas spp., Eubacterium spp., Sneathia spp./Leptotrihia spp./Fusobacterium spp., Megasphaera spp./Veilonella spp./Dialister spp., Lachnobacterium spp./Clostridium spp., Mobiluncus spp./Corynebacterium spp., Peptostreptococcus spp., Atopobium vaginae, Mycoplasma hominis, Ureaplasma (urealyticum+parvum), Mycoplasma genitalium, and Candida spp. The Femoflor® Screen Real-Time PCR Detection Kit was designed to target one or more of the following characteristics or strains: total bacterial mass (TBM), Lactobacillus spp., Gardnerella vaginalis/Prevotella bivia/Porphyromonas spp., Candida spp., Mycoplasma hominis, Ureaplasma (urealyticum+parvum), Mycoplasma genitalium, Trichomonas vaginalis, Neisseria gonorrhoeae, Chlamydia trachomatis, HSV-2, CMV, and HSV-1. Two strips were used for one sample test, e.g., 8 strips were used to analyze 2 samples-4 for the test samples, 2 for positive control and 2 for negative control. Each strip was placed in a plastic tube for further analysis.

Next, the Taq-polymerase solution from the Femoflor® 16 Real-Time PCR Detection Kit or the MAX Taq-polymerase solution from the Femoflor® Screen Real-Time PCR Detection Kit was vortexed thoroughly (3-5 sec), and centrifuged briefly (1-3 sec). 10 μl of Taq-polymerase solution or MAX Taq-polymerase solution and 20 μl of mineral oil (one drop from 100-1000 μl tip approximately) were added into each tube. Next, 5.0 μl of DNA sample was added into each tube and the tubes were spun briefly (1-3 sec). The tubes were then placed in a real-time PCR instrument and the following cycle program was used: an initial step of 30 seconds of 80° C. and 1 minute 30 second of 94° C., 5 cycles of 30 seconds at 94° C., 15 seconds at 64° C., or 45 cycles of 10 seconds at 94° C., 15 seconds of 64° C., and the last cycle of 5 minutes at 94° C. The resulting products were stored at 10° C. before retrieved from the instruments for longer storage or further analyses. After storage, the products were analyzed as follows.

### Femoflor® Assay Data Collection and Analysis

The optical unit of a real-time PCR instrument collects data automatically as the cycling program proceeds. A qualitative analysis was performed for pathogenic bacteria. Specifically, for each sample, the following panels were evaluated: (i) presence/absence Mycoplasma genitalium; (ii) the amount of human genomic DNA (SIC—sample intake control), total number of all bacteria (total bacterial mass—TBM), amount of lactobacilli, commensals, opportunistic bacteria; and (iii) amount of Candida spp. The absolute number of microorganisms was evaluated in genome equivalents (GE), the number of which is proportional to the microbial contamination of the biotope from which the biomaterial sample was obtained.

### Example 1

### Biological Samples Collected by Menstrual Tampon and Femoflor® 16 Assay

This example presents the results from a study to compare a menstrual tampon and vaginal swab as sample collection methods prior to testing with the Femoflor® 16 assay.

CBD-containing tampons (Daye, London) were self-administered to the vagina by 11 volunteers for 20 minutes and collected. Vaginal swab samples were also taken from the volunteers by an obstetrician/gynecologist. Total DNA was isolated from the samples using the PREP-NA-PLUS kit and methods described above. Next, the samples were evaluated using the Femoflor® 16 assay and methods described above. For each volunteer, the results generated by the different vaginal collection methods were compared and dysbiosis or normocenosis was evaluated using the Femoflor® 16 assay PCR analysis software and its underlying algorithm.

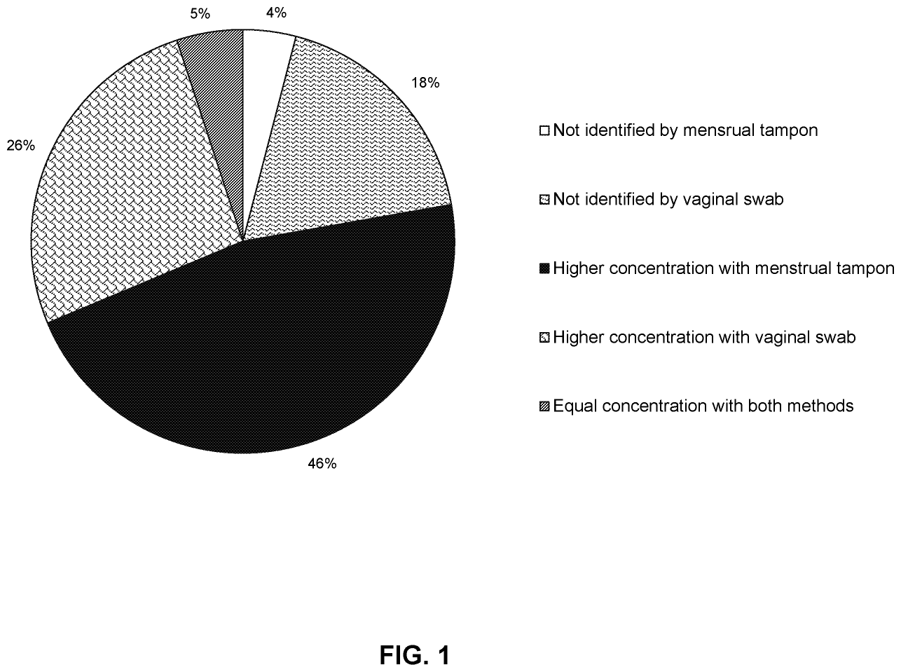

Nearly half of the bacterial panels identified from the samples showed a higher bacterial concentration when a tampon was used for sample collection compared to a vaginal swab (FIG. 1). The pie chart of FIG. 1 shows the percentage of samples having certain characteristics, such as the percentage of samples where a higher bacterial concentration was observed using a tampon for sample collection and the percentage of samples where a microorganism was not detected using a tampon for sample collection. In particular, these results show nearly half of the bacterial panels identified from the samples showed a higher bacterial concentration when a tampon was used for sample collection compared to a vaginal swab.

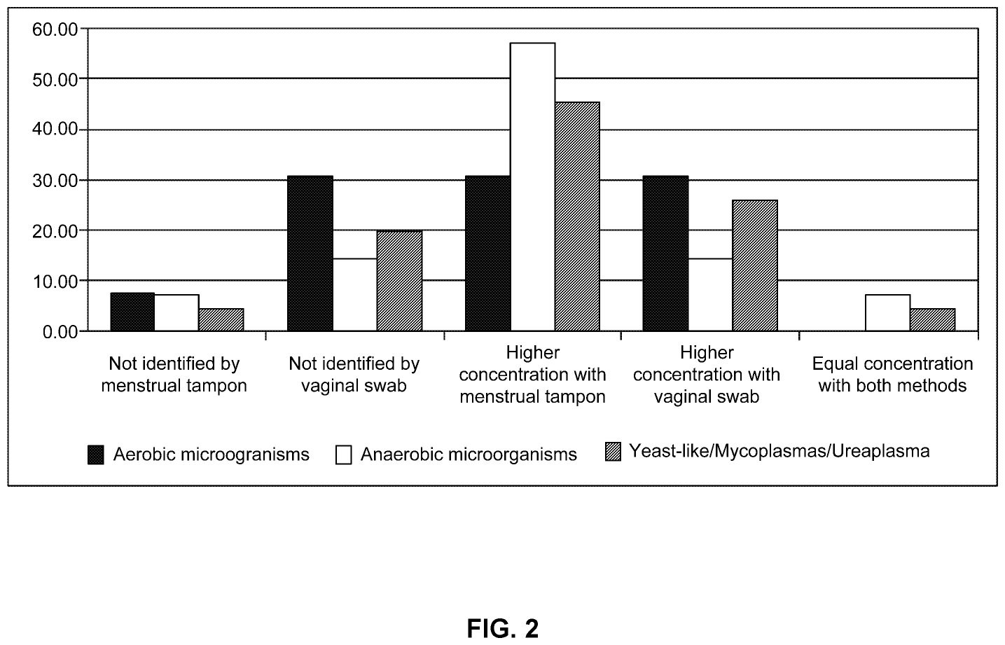

In all volunteers and panels tested, only one of the pathogens (grouped together in Yeast-like/Mycoplasmas/Ureaplasma) was not identified in a sample from a tampon (FIG. 2). As such, samples collected from a vaginal tampon gave much better bacterial amplification and identification compared to samples collected from a vaginal swab.

In addition, approximately 18% of bacterial panels identified from samples collected from a tampon were not detected in samples collected from a vaginal swab (FIG. 1). In such cases, collection of the sample with a vaginal swab appeared to give a false negative result for the volunteer.

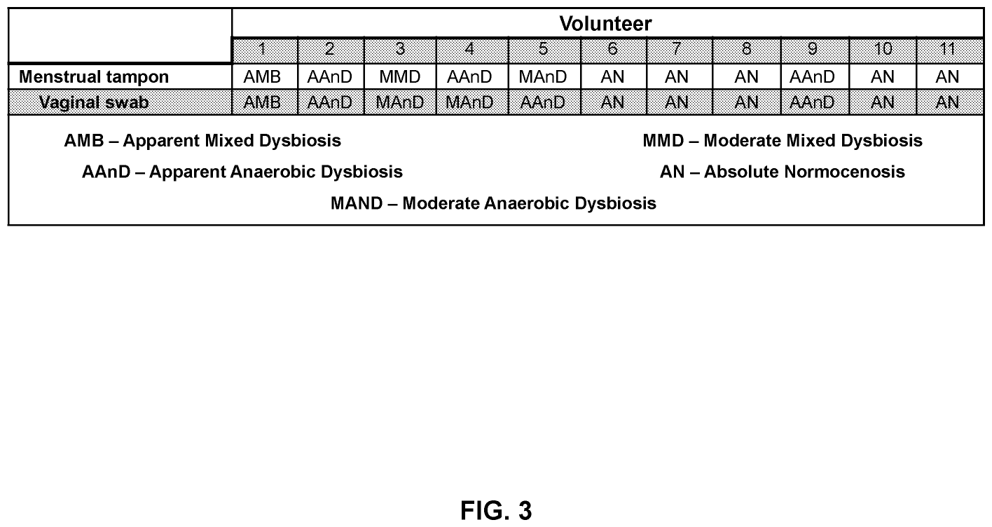

Moreover, 3 of the 11 volunteers showed different conclusions with regard to the level of severity of dysbiosis for the different sample collection methods (FIG. 3). In two instances, the difference was in the severity of the dysbiosis—Moderate vs Apparent. Specifically, one volunteer showed a moderate dysbiosis based on a tampon sample, and the other showed it from a swab sample. Additionally, Volunteer 3 showed MMD (Moderate Mixed Dysbiosis) based on a tampon sample, and a MAnD (Moderate Anaerobic Dysbiosis) based on a swab sample. In this case, the difference in diagnosis could be attributed to the fact that 2 groups of obligative anaerobes were not identified from the tampon sample.

### Example 2

### Biological Samples Collected by Menstrual Tampon and Femoflor® Screen Assay

This example presents the results from a further study to compare a menstrual tampon, cervical swab and vaginal swab as sample collection methods prior to testing with the Femoflor® Screen assay.

CBD-containing tampons (Daye, London) were self-administered to the vagina by 23 patients for 20 minutes and collected. Vaginal swab and cervical swab samples were also taken from the patients by an obstetrician/gynecologist (OBGYN). Total DNA was isolated from the samples using the PREP-NA-PLUS kit and methods described above. Next, the samples were evaluated using the Femoflor® Screen assay and methods described above. For each patient, the results generated from the three different collection methods were compared, and dysbiosis or normocenosis was evaluated by an OBGYN.

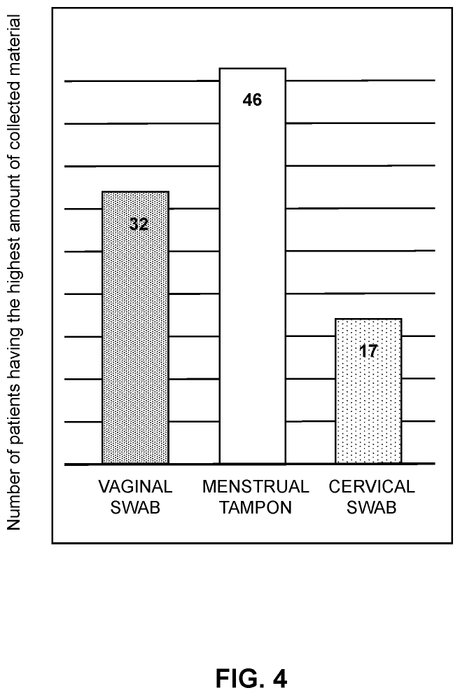

The results of this study show that 45% of patients had the highest amount of biological material collected with a menstrual tampon (46 out of 102), compared to 31% of patients having the highest amount of biological material collected with a vaginal swab (32 out of 102), and 17% of patients having the highest amount of biological material collected with a cervical swab (17 out of 102) (FIG. 4).

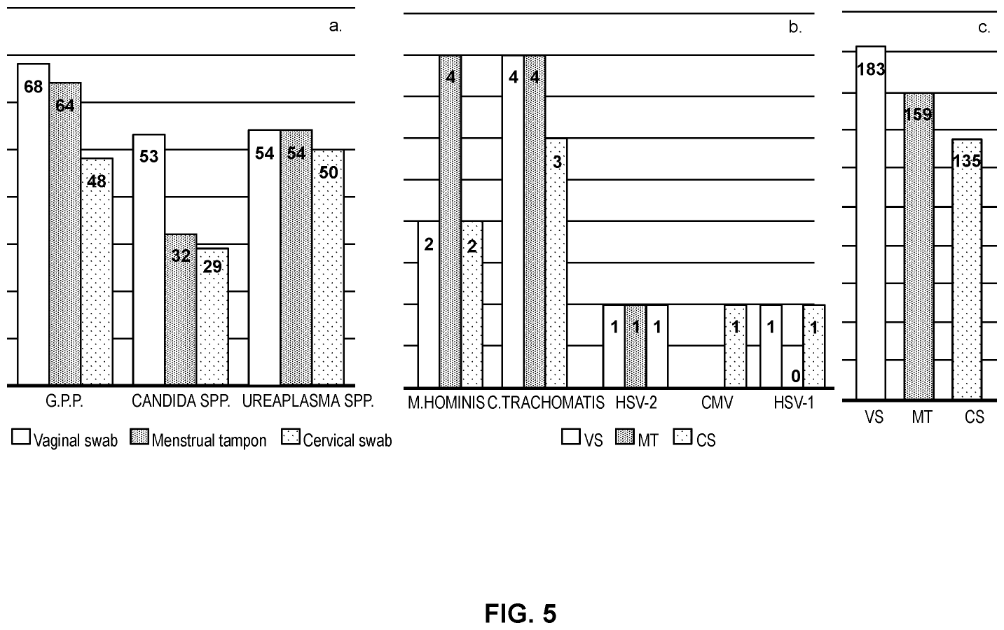

Results from the Femoflor® Screen assay are shown in FIG. 5. Specifically,

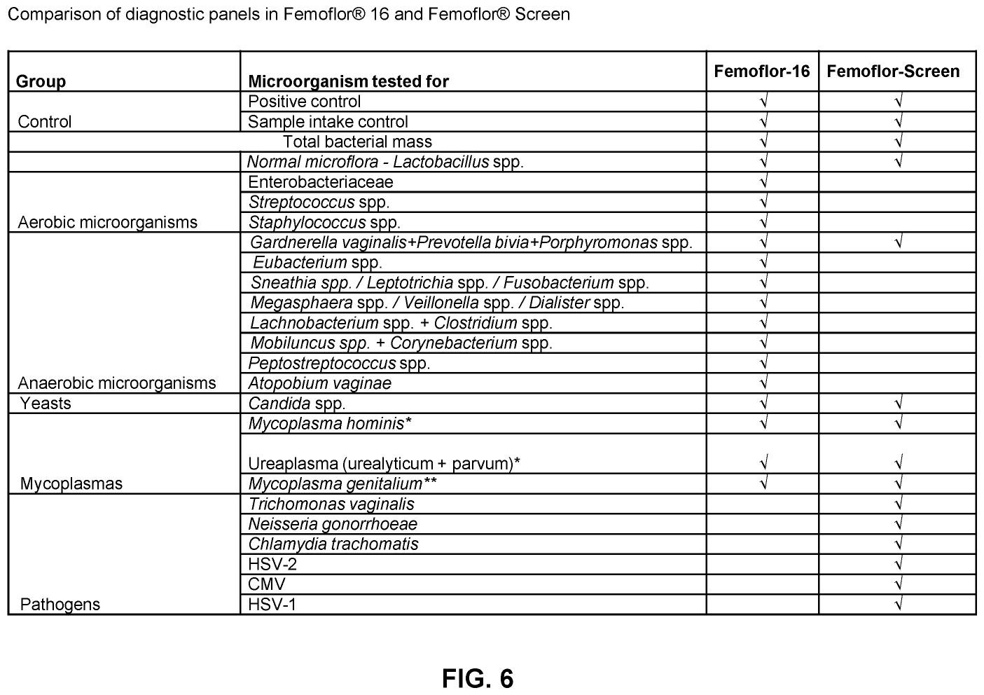

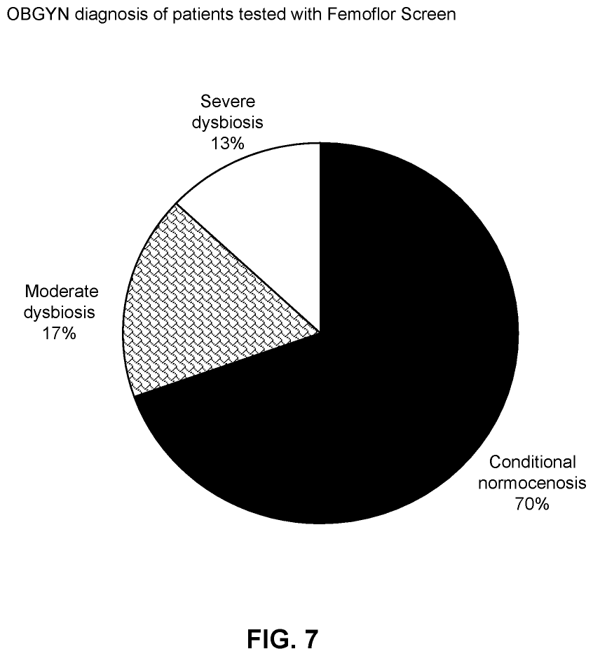

FIG. 5(a) and (b) show the numbers of identified microorganism species using each sample collection method. FIG. 5(c) shows the total number of identified microorganism species using each sample collection method. The diagnostic panels of Femoflor® 16 and Femoflor® Screen were compared and tabulated to illustrate the differences in testing capacities (FIG. 6). Only two patients were diagnosed differently among the sample collection methods. For the first patient, moderate dysbiosis was diagnosed based on analysis of a sample collected using a vaginal swab, whereas conditional normocenosis was diagnosed based on analysis of samples collected using a menstrual tampon and cervical swab. For the second patient, conditional normocenosis was diagnosed based on analysis of samples collected using a vaginal swab and menstrual tampon, whereas moderate dysbiosis was diagnosed based on analysis of a sample collected by a cervical swab. Furthermore, in another patient, a diagnosis difference was observed only in the degree of the condition. Specifically, analysis of samples collected using a menstrual tampon and vaginal swab led to a diagnosis of severe dysbiosis, whereas analysis of a sample collected using a cervical swab led to a diagnosis of moderate dysbiosis. An overview of the most severe OBGYN diagnosis among three sampling collection methods of all patients is shown in FIG. 7. Conditional normocenosis was most frequently diagnosed as the most severe (70%), followed by moderate dysbiosis (17%), and severe dysbiosis (13%).

Overall, these results show that biological sample collection with a tampon provides at least the same microorganism detection and diagnosis compared to samples collected by a conventional vaginal swab or cervical swab. As such, these results demonstrate that a tampon is a reliable sample collection tool for evaluating the vaginal microbiome composition or testing for vaginal or cervical microorganisms or infection (e.g., sexually transmitted infections). Moreover, the ability to self-sample and collect biological samples with a tampon would encourage more women to monitor their vaginal health.

What is claimed is:

1. A method for obtaining a biological sample from a subject, comprising obtaining a biological sample from a tampon comprising (i) an absorbent tampon body and (ii) a protective sock around the tampon body; wherein said tampon having been administered to the subject vaginally for a time sufficient to obtain a biological sample on the protective sock.

2. A method for obtaining a biological sample from a subject, comprising:
(a) obtaining a tampon comprising (i) an absorbent tampon body and (ii) a protective sock around the tampon body; wherein said tampon having been administered to the subject vaginally for a time sufficient to obtain a biological sample on the protective sock; and
(b) obtaining the biological sample from the protective sock.

3. The method of claim 1 or 2, further comprising separating the protective sock from the tampon before obtaining the biological sample from the protective sock.

4. A method for obtaining a biological sample from a subject, comprising:
(a) administering a tampon to the subject vaginally; wherein the tampon comprises (i) an absorbent tampon body and (ii) a protective sock around the tampon body; and
(b) maintaining the tampon in the vagina for a time sufficient to obtain a biological sample on the protective sock.

5. The method of claim 4, further comprising (c) removing the tampon from the vagina.

6. The method of claim 5, further comprising (d) separating the protective sock from the tampon.

7. The method of claim 6, further comprising (e) obtaining the biological sample from the protective sock.

8. The method of claim 7, wherein the protective sock is made of industrial hemp fiber.

9. The method of claim 7, wherein the protective sock is made of rayon, cotton, or a mixture thereof

10. The method of any one of claims 1-9, wherein the tampon body is made of industrial hemp fiber.

11. The method of any one of claims 1-9, wherein the tampon body is made of rayon, cotton, or a mixture thereof.

12. The method of any one of claims 1-11, wherein the tampon further comprises a pharmaceutical composition comprising an effective amount of cannabidiol (CBD).

13. The method of any one of claims 1-12, wherein the tampon is maintained in the vagina for at least 20 minutes.

14. The method of any one of claims 1-13, wherein the biological sample is obtained at least 5 days after the tampon is removed from the vagina.

15. The method of any one of claims 1-14, wherein the protective sock is stored at room temperature after the tampon is removed from the vagina.

16. A method to detect a sexually transmitted infection (STI) in a subject, comprising performing a nucleic acid assay on a biological sample to detect and/or quantify the presence of an STI marker; wherein said detecting and/or quantifying of the marker is indicative of an STI in the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

17. A method to detect an STI in a subject, comprising:
(a) obtaining a tampon comprising (i) an absorbent tampon body and (ii) a protective sock around the tampon body; wherein said tampon having been administered to the subject vaginally for a time sufficient to obtain a biological sample on the protective sock; and
(b) performing a nucleic acid assay on the biological sample to detect and/or quantify the presence of an STI marker;
wherein said detecting and/or quantifying of the marker is indicative of an STI in the subject.

18. A method to detect an STI in a subject, comprising:
(a) administering a tampon to the subject vaginally; wherein the tampon comprises (i) an absorbent tampon body and (ii) a protective sock around the tampon body;
(b) maintaining the tampon in the vagina for a time sufficient to obtain a biological sample on the protective sock; and
(c) performing a nucleic acid assay on the biological sample to detect and/or quantify the presence of an STI marker;
wherein said detecting and/or quantifying of the marker is indicative of an STI in the subject.

19. The method of any one of claims 16-18, further comprising separating the protective sock from the tampon before performing the nucleic acid assay.

20. The method of any one of claims 16-18, further comprising isolating the biological sample from the protective sock before performing the nucleic acid assay.

21. The method of any one of claims 16-20, wherein the nucleic acid assay is polymerase chain reaction (PCR).

22. The method of claim 21, wherein the PCR is real-time PCR or quantitative PCR.

23. The method of claim 21 or 22, wherein the PCR comprises isolation of DNA from the biological sample, amplification of a DNA sequence of an STI marker, and detection and/or quantification of the amplified sequence.

24. The method of any one of claims 21-23, wherein the PCR further comprises comparison of the quantified amount of the STI marker to a reference amount of the STI marker.

25. The method of claim 24, wherein the STI marker is an aerobic microorganism.

26. The method of claim 24, wherein the STI marker is an anaerobic microorganism.

27. The method of claim 24, wherein the STI marker is a bacteria, virus, yeast, mycoplasma, or ureaplasma.

28. The method of claim 24, wherein the STI marker is a bacteria of the phyla Firmicutes, Actinobacteria, Bacteroidetes, Fusobacteria, or Proteobacteria.

29. The method of claim 24, wherein the STI is chlamydia, herpes, genital herpes, HSV-1, HSV-2, hepatitis B, trichomoniasis, human immunodeficiency virus (HIV), acquired immunodeficiency syndrome (AIDS), human papilloma virus (HPV), CMV, chancroid, molluscum contagiosum, scabies, syphilis, gonorrhea, or crabs.

30. The method of any one of claims 16-29, wherein the protective sock is made of industrial hemp fiber.

31. The method of any one of claims 16-29, wherein the protective sock is made of rayon, cotton, or a mixture thereof.

32. The method of any one of claims 16-31, wherein the tampon body is made of industrial hemp fiber.

33. The method of any one of claims 16-31, wherein the tampon body is made of rayon, cotton, or a mixture thereof.

34. The method of any one of claims 16-33, wherein the tampon further comprises a pharmaceutical composition comprising an effective amount of CBD.

35. The method of any one of claims 16-34, wherein the tampon is maintained in the vagina for at least 20 minutes.

36. The method of any one of claims 16-35, wherein the biological sample is obtained at least 5 days after the tampon is removed from the vagina.

37. The method of any one of claims 16-36, wherein the protective sock is stored at room temperature after the tampon is removed from the vagina.

38. A method to determine the composition of the vaginal microbiome of a subject, comprising performing a nucleic acid assay on a biological sample to determine and/or quantify the vaginal microbiome of the subject; wherein the dominant species of bacteria in the biological sample is indicative of the composition of the vaginal microbiome of the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

39. A method to determine the composition of the vaginal microbiome of a subject, comprising:
(a) obtaining a tampon comprising (i) an absorbent tampon body and (ii) a protective sock around the tampon body; wherein said tampon having been administered to the subject vaginally for a time sufficient to obtain a biological sample on the protective sock; and
(b) performing a nucleic acid assay on the biological sample to determine and/or quantify the vaginal microbiome of the subject; wherein the dominant species of bacteria in the biological sample is indicative of the composition of the vaginal microbiome of the subject.

40. A method to determine the composition of the vaginal microbiome of a subject, comprising:
(a) administering a tampon to the subject vaginally; wherein the tampon comprises (i) an absorbent tampon body and (ii) a protective sock around the tampon body;
(b) maintaining the tampon in the vagina for a time sufficient to obtain a biological sample on the protective sock; and
(c) performing a nucleic acid assay on the biological sample to determine and/or quantify the vaginal microbiome of the subject;
wherein the dominant species of bacteria in the biological sample is indicative of the composition of the vaginal microbiome of the subject.

41. The method of any one of claims 38-40, further comprising separating the protective sock from the tampon before performing the nucleic acid assay.

42. The method of any one of claims 38-41, further comprising isolating the biological sample from the protective sock before performing the nucleic acid assay.

43. The method of any one of claims 38-42, wherein the nucleic acid assay is PCR.

44. The method of claim 43, wherein the PCR is real-time PCR or quantitative PCR.

45. The method of claim 43 or 44, wherein the PCR comprises isolation of DNA from the biological sample, amplification of total bacterial DNA from the biological sample, and detection and/or quantification of the dominant species of bacteria in the biological sample.

46. The method of any one of claims 38-45, wherein the protective sock is made of industrial hemp fiber.

47. The method of any one of claims 38-45, wherein the protective sock is made of rayon, cotton, or a mixture thereof.

48. The method of any one of claims 38-47, wherein the tampon body is made of industrial hemp fiber.

49. The method of any one of claims 38-47, wherein the tampon body is made of rayon, cotton, or a mixture thereof.

50. The method of any one of claims 38-49, wherein the tampon further comprises a pharmaceutical composition comprising an effective amount of CBD.

51. The method of any one of claims 38-50, wherein the tampon is maintained in the vagina for at least 20 minutes.

52. The method of any one of claims 38-51, wherein the biological sample is obtained at least 5 days after the tampon is removed from the vagina.

53. The method of any one of claims 38-52, wherein the protective sock is stored at room temperature after the tampon is removed from the vagina.

54. A method to detect dysbiosis in a subject, comprising performing a nucleic acid assay on a biological sample to detect dysbiosis in the subject; wherein the biological sample is present on a tampon comprising (i) an absorbent tampon body and (ii) a protective sock around the tampon body; and wherein said tampon having been administered to the subject vaginally for a time sufficient to obtain a biological sample on the protective sock.

55. A method to detect dysbiosis in a subject, comprising:
(a) obtaining a tampon comprising (i) an absorbent tampon body and (ii) a protective sock around the tampon body; wherein said tampon having been administered to the subject vaginally for a time sufficient to obtain a biological sample on the protective sock; and
(b) performing a nucleic acid assay on the biological sample to detect dysbiosis in the subject.

56. A method to detect dysbiosis in a subject, comprising:
(a) administering a tampon to the subject vaginally; wherein the tampon comprises (i) an absorbent tampon body and (ii) a protective sock around the tampon body;
(b) maintaining the tampon in the vagina for a time sufficient to obtain a biological sample on the protective sock; and
(c) performing a nucleic acid assay on the biological sample to detect dysbiosis in the subject.

57. The method of any one of claims 54-56, further comprising separating the protective sock from the tampon before performing the nucleic acid assay.

58. The method of any one of claims 54-57, further comprising isolating the biological sample from the protective sock before performing the nucleic acid assay.

59. The method of any one of claims 54-58, wherein the nucleic acid assay is PCR.

60. The method of claim 59, wherein the PCR is real-time PCR or quantitative PCR.

61. The method of claim 59 or 60, wherein the PCR comprises isolation of DNA from the biological sample, amplification of a DNA sequence of a marker for dysbiosis, and detection and/or quantification of the amplified sequence.

62. The method of any one of claims 59-61, wherein the PCR further comprises comparison of the quantified amount of the amplified sequence of the dysbiosis marker to a reference amount of the dysbiosis marker.

63. The method of any one of claims 54-62, wherein dysbiosis is indicated in the subject when less than about 80% of bacteria in the biological sample is Lactobacillus.

64. The method of claim 63, wherein the Lactobacillus is L. crispatus, L. iners, L. gasseri, or L. jensenii.

65. The method of any one of claims 54-64, wherein the dysbiosis is moderate dysbiosis.

66. The method of claim 65, wherein moderate dysbiosis is indicated in the subject when from about 20% to about 80% of bacteria in the biological sample is Lactobacillus.

67. The method of any one of claims 54-64, wherein the dysbiosis is apparent dysbiosis.

68. The method of claim 67, wherein apparent dysbiosis is indicated in the subject when less than about 20% of bacteria in the biological sample is Lactobacillus.

69. The method of any one of claims 54-64, wherein the dysbiosis is apparent mixed dysbiosis, moderate mixed dysbiosis, apparent anaerobic dysbiosis, or moderate anaerobic dysbiosis.

70. The method of any one of claims 54-69, wherein the protective sock is made of industrial hemp fiber.

71. The method of any one of claims 54-69, wherein the protective sock is made of rayon, cotton, or a mixture thereof.

72. The method of any one of claims 54-71, wherein the tampon body is made of industrial hemp fiber.

73. The method of any one of claims 54-71, wherein the tampon body is made of rayon, cotton, or a mixture thereof.

74. The method of any one of claims 54-73, wherein the tampon further comprises a pharmaceutical composition comprising an effective amount of CBD.

75. The method of any one of claims 54-74, wherein the tampon is maintained in the vagina for at least 20 minutes.

76. The method of any one of claims 54-75, wherein the biological sample is obtained at least 5 days after the tampon is removed from the vagina.

77. The method of any one of claims 54-76, wherein the protective sock is stored at room temperature after the tampon is removed from the vagina.

78. A kit, comprising:
(a) a tampon comprising (i) an absorbent tampon body and (ii) a protective sock around the tampon body; and
(b) instructions for obtaining a biological sample with the tampon.

79. The kit of claim 78, further comprising a container for storing the tampon after the biological sample is collected.

80. The kit of claim 78 or 79, further comprising a self-assessment survey.

81. The kit of any one of claims 78-80, further comprising a mailing envelope for shipping the tampon after the biological sample is collected.

82. The kit of any one of claims 78-81, wherein the protective sock is made of industrial hemp fiber.

83. The kit of any one of claims 78-81, wherein the protective sock is made of rayon, cotton, or a mixture thereof.

84. The kit of any one of claims 78-83, wherein the tampon body is made of industrial hemp fiber.

85. The kit of any one of claims 78-83, wherein the tampon body is made of rayon, cotton, or a mixture thereof.

86. The kit of any one of claims 78-85, wherein the tampon further comprises a pharmaceutical composition comprising an effective amount of CBD.
